(19) United States
(12) Patent Application Publication
(10) Pub. No.: US 2015/0126889 A1
(43) Pub. Date: May 7, 2015

(54) SPIROMETER COMPRISING PIEZOELECTRIC SENSOR

(71) Applicant: South Jersey Engineering & Research, LLC, Avalon, NJ (US)

(72) Inventors: Jonathan Mack Frey, Avalon, NJ (US); Charles Lee Frey, Melbourne Beach, FL (US)

(21) Appl. No.: 14/458,863

(22) Filed: Aug. 13, 2014

Related U.S. Application Data
(60) Provisional application No. 61/899,736, filed on Nov. 4, 2013.
(60) Provisional application No. 61/931,917, filed on Jan. 27, 2014.

(51) Int. Cl. A61B 5/087; G01F 1/32; A61B 5/00

(52) U.S. Cl. CPC A61B 5/087; A61B 5/742; G01F 1/3209
USPC 600/538

(57) ABSTRACT

The present invention is directed to a spirometer comprising a piezoelectric sensor and the use of the spirometer in measuring a user's lung performance and/or tracking a user's lung performance over a period of time. The spirometer is configured so that fluid flow through a housing produces oscillating stresses in a piezoelectric material. The oscillating stresses produce an electric signal. Characteristics of the electric signal, such as the magnitude of the signal at particular frequencies, can be measured and used to determine the rate of fluid flow through the housing during inhalation or exhalation. The fluid flow characteristics may then be displayed on a variety of devices, such as a smartphone, a personal computer, etc.

This application claims priority to U.S. Provisional Application No. 61/899,736, filed on Nov. 4, 2013 and to U.S. Provisional Application No. 61/931,917, filed on Jan. 27, 2014. Each of the above-identified applications is hereby incorporated by reference in its entirety.

### BACKGROUND OF THE INVENTION

1. Field of the Invention

Spirometers are devices that are configured to measure the flow of air inhaled and exhaled by a user. This information is useful in testing a user's lung performance and, accordingly, lung health. Spirometers are commonly used as part of pulmonary function testing and to evaluate lung function in people with obstructive or restrictive lung disease such as asthma. Spirometers are also used to study the progress of a patient's lung performance to assist in the treatment of a variety of diseases and conditions.

2. Description of the Related Art

Spirometers are often used in a health care setting to perform a number of different tests relating to lung performance. For example, a patient may be asked to take the deepest breath they can, and then exhale into the spirometer as hard as possible, for as long as possible, and preferably for at least 6 seconds. This forced exhalation is sometimes directly followed or preceded by a rapid inhalation. This test may also be preceded by a period of quiet breathing in and out of the spirometer in order to determine the tidal volume. The measurements from the spirometer are then used to calculate any of a number of different data sets.

Many conventional spirometers evaluate the flow of air by measuring the pressure difference before and after a membrane or capillaries having a known resistance. The signal is then converted into a voltage in order to create electronic data, which can be displayed on a monitor. Other conventional spirometers evaluate the flow of air by measuring the rotations of a turbine, wherein the speed of rotation of the turbine corresponds to the velocity of the air flow. Typically, an infrared detector detects the rate at which the light from an infrared source is interrupted by the passing of the turbine. The signal is then converted into a voltage in order to create electronic data, which can be displayed on a monitor.

Another type of spirometer is one that is used to improve the performance of a user's lungs, commonly known as an incentive spirometer. Typically, an incentive spirometer is used by medical patients recovering from surgery or otherwise requiring extended in-bed recovery. To use an incentive spirometer, the patient breathes in from the device slowly and deeply, then holds his or her breathe for a number of seconds. An indicator, such as a plunger, moves in response to the patient's inhalation vacuum. The movement of the plunger to a sustained position is measured by a scale printed on the device and/or against a goal marker. The patient is generally asked to do many repetitions a day while keeping track of his or her progress.

### SUMMARY OF THE INVENTION

The present invention is directed to a spirometer comprising a piezoelectric sensor and the use of the spirometer in measuring a user's lung performance and/or tracking a user's lung performance over a period of time. When compared with conventional spirometers, the spirometer of embodiments of the present invention provides improvements in performance, durability, portability, ease of use, and cost.

One aspect of the invention is directed to a spirometer for measuring lung performance comprising a housing having at least a first fluid opening and a second fluid opening and a fluid flow sensor comprising a piezoelectric material oriented within the housing to produce an electric signal in response to fluid flow through the housing. The spirometer is configured so that fluid flow through the housing produces oscillating stresses in the piezoelectric material and the resulting electric signal has a magnitude that corresponds with the rate of fluid flow through the housing.

Another aspect of the invention is directed to a spirometer for measuring lung performance comprising a housing having at least a first fluid opening and a second fluid opening and a fluid flow sensor that includes at least a cantilever comprising a piezoelectric material that is operable to produce an electric signal in response to stresses in the material. The spirometer is configured so that fluid flow through the housing acts on the fluid flow sensor so as to cause movement of the attached cantilever in an oscillating manner, thereby producing an electric signal having a measurable magnitude that corresponds with the rate of fluid flow through the housing.

Another aspect of the invention is directed to a spirometer for measuring lung performance comprising a housing having at least a first fluid opening and a second fluid opening and a fluid flow sensor comprising a piezoelectric material oriented within the housing to produce an electric signal in response to fluid flow through the housing. The spirometer is configured to produce a structured flow of fluid over the sensor, such that the amplitude of the electric signal at a particular frequency or the magnitude of the electric signal at a particular set of frequencies closely corresponds with the rate of fluid flow through the housing. By producing a structured flow of fluid over the sensor, the spirometer provides for a precise measurement of lung performance that is independent of various external factors.

Another aspect of the invention is directed to a spirometer comprising a housing having at least a first fluid opening and a second fluid opening and a fluid flow sensor comprising a piezoelectric material oriented within the housing to produce an electric signal in response to fluid flow through the housing. The spirometer is configured so that fluid flow through the housing produces oscillating stresses in the piezoelectric material and the resulting electric signal has a magnitude that corresponds with the rate of fluid flow through the housing. The spirometer is also configured to be coupled to an external display device, such as a smartphone or personal computer, by a physical and/or wireless connection.

Another aspect of the invention is directed to a spirometer comprising a housing having at least a first fluid opening and a second fluid opening and a fluid flow sensor that includes at least a cantilever comprising a piezoelectric material that is operable to produce an electric signal in response to stresses in the material and a stimulator that is operable to induce movement of the cantilever and a corresponding stressing of the piezoelectric material in response to fluid flow through the housing. For instance, fluid flow through the housing may interact with the stimulator to produce vortex shedding, which causes movement of the attached cantilever in an oscillating manner. The spirometer is also configured to be coupled to an external display device, such as a smartphone or personal computer, by a physical or wireless connection.

Another aspect of the invention is directed to a spirometer comprising a housing having at least a first fluid opening and a second fluid opening and a fluid flow sensor comprising a piezoelectric material oriented within the housing to produce an electric signal in response to fluid flow through the housing. The spirometer is configured to produce a structured flow of fluid over the sensor, such that the magnitude of the electric signal at a predetermined band of frequencies corresponds with the rate of fluid flow through the housing. The spirometer is also configured to be coupled to an external display device, such as a smartphone or personal computer, by a physical or wireless connection.

Another aspect of the invention is directed to a method for measuring lung performance by inhaling or exhaling into the spirometer of at least one embodiment of the invention. In various embodiments, the output data may be displayed on a personal computer or smartphone and the lung performance data may be tracked over a period of time. For instance, at least one of the electric signal and the output data may be transmitted to an external display, such as a personal computer or a smartphone, using either a physical connection or a wireless connection.

For a better understanding of the invention, its operating advantages, and the specific objects attained by its uses, reference should be had to the accompanying drawings and descriptive matter in which there is illustrated an exemplary embodiment of the invention.

### BRIEF DESCRIPTION OF THE DRAWINGS

A clear conception of the advantages and features of one or more embodiments will become more readily apparent by reference to the exemplary, and therefore non-limiting, embodiments illustrated in the drawings:

FIG. 1 is a perspective view, in section, of an embodiment of the spirometer, in which the fluid flow sensor includes a stimulator.

FIG. 2 is a perspective view of an embodiment of the fluid flow sensor showing the flexing of the cantilever arm in a first direction in response to fluid flow through the spirometer.

FIG. 3 is a perspective view of an embodiment of the fluid flow sensor showing the flexing of the cantilever arm in a second direction in response to fluid flow through the spirometer.

FIG. 4 is a flow diagram showing the conversion of an electric signal in the fluid flow sensor to output data, according to an embodiment of the present invention.

FIG. 5 is an exploded perspective view of an embodiment of the spirometer comprising a column or series of columns that is configured to produce a structured fluid flow at the fluid flow sensor.

FIG. 6 is an exploded perspective view of an embodiment of the spirometer comprising an inverted column or series of inverted columns that is configured to produce a structured fluid flow at the fluid flow sensor.

FIG. 7 is an exploded perspective view of an embodiment of the spirometer comprising a fluid flow conditioner between the first fluid opening and the fluid flow sensor.

FIG. 8 is an exploded perspective view of an embodiment of the spirometer comprising a first fluid flow conditioner between the first fluid opening and the fluid flow sensor and a second fluid flow conditioner between the second fluid opening and the fluid flow sensor.

FIG. 9 is an exploded perspective view of an embodiment of the spirometer comprising both a fluid flow conditioner and a velocity enhancer between the first fluid opening and the fluid flow sensor

FIG. 10 is an exploded perspective view of an embodiment of the spirometer comprising contoured walls that are configured to produce a structured flow at the fluid flow sensor.

FIG. 11 is an image of an exploded side view of an embodiment of the spirometer.

FIG. 12 is an image of a perspective view of an embodiment of the spirometer.

FIG. 13 is an image of an end view of an embodiment of the spirometer.

FIG. 14 is an image of a perspective view of an embodiment of the spirometer.

FIG. 15 is a graph showing the accuracy of an embodiment of the calibrated spirometer.

FIG. 16 is a graph of normalized data showing the accuracy of embodiments of the calibrated spirometer.

### DETAILED DESCRIPTION OF THE INVENTION

Embodiments of the present invention are directed to a spirometer 1 that utilizes the oscillating stresses placed upon a piezoelectric material in response to vortex shedding. Vortex shedding is an oscillating flow that may take place when a fluid such as air or water flows past an object to create low-pressure vortices at the downstream side of the object. The low-pressure vortices are shed from alternating sides of the object, creating periodic lateral forces on the object and causing it to vibrate. If the vortex shedding frequency is similar to the natural frequency of the object, it causes resonance. Vortex shedding may cause an object that is not rigidly mounted, such as a cantilever, to oscillate in a direction lateral to the fluid flow.

By producing a spirometer 1 that relies on vortex shedding to produce oscillating stresses in a piezoelectric material, embodiments of the present invention provide a highly effective, low-cost, and extremely portable spirometer that has a number of benefits over those known in the art.

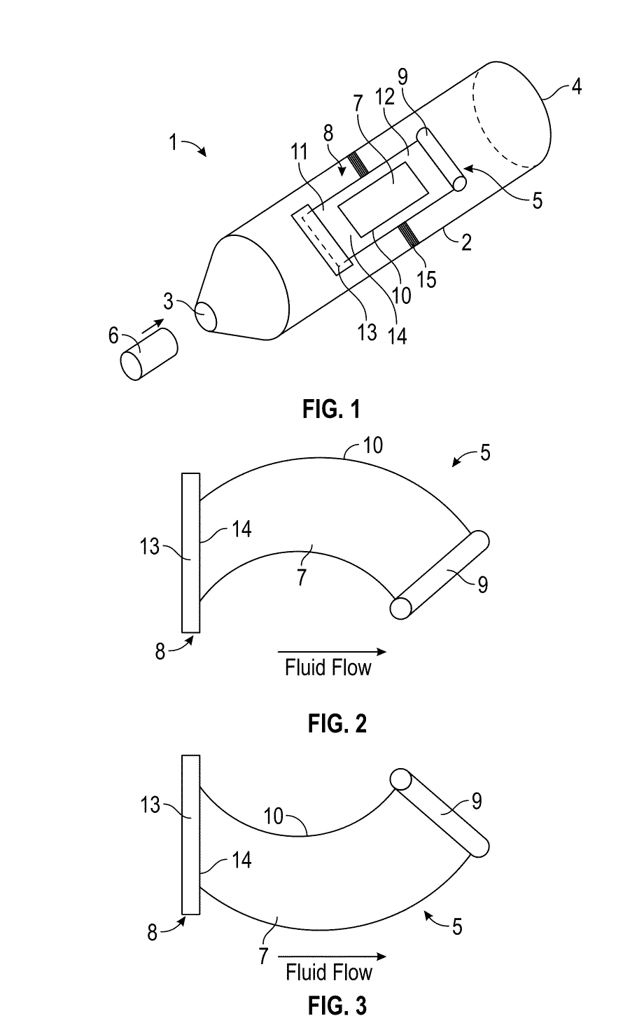

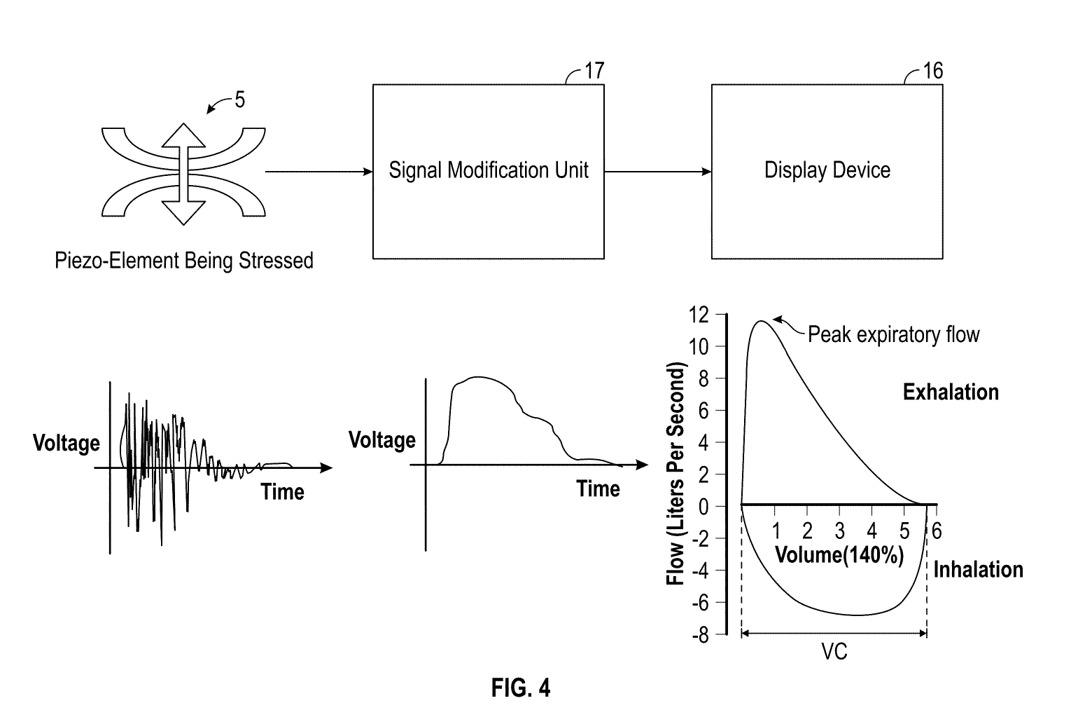

The spirometer 1 comprises a housing 2. Although the housing 2 of the exemplary embodiment illustrated in FIG. 1 is in the shape of a tube, the housing may take any shape. The housing 2 may be configured so as to be portable. For example, the housing 2 may be made of a durable material or may be configured to have a size and shape that fits easily into a protective pouch or pocket.

The housing 2 comprises at least a first fluid opening 3 and a second fluid opening 4. The first fluid opening 3 and second fluid opening 4 are located such that fluid flowing between the two openings interacts with a fluid flow sensor 5. In the exemplary embodiment illustrated in FIG. 1, the first fluid opening 3 and second fluid opening 4 are located at opposite ends of the housing 1. Other arrangements of the fluid openings are also contemplated, however, so long as fluid flow between the first fluid opening 3 and the second fluid opening 4 interacts with a fluid flow sensor 5.

The first fluid opening 3 is configured for the user to inhale or exhale air through the housing 2. Although the direction of air flow through the first fluid opening 3 may vary depending on the use of the spirometer (i.e. whether the user is inhaling or exhaling), this opening will also be referred to as the inlet 3.

In embodiments, the inlet 3 is configured such that a user can comfortably and effectively inhale and/or exhale through the fluid opening and into the housing 2. For example, in at least one embodiment, the inlet 3 comprises a mouthpiece 6. The mouthpiece 6 may be a disposable mouthpiece or a reusable mouthpiece. When designed for home use, a reusable mouthpiece may be preferred. The reusable mouthpiece may be removable from the housing 2. By removing the mouthpiece 6 from the housing 2, cleaning of the mouthpiece may be simplified. The reusable mouthpiece may also be non-removable. For example, the reusable mouthpiece may be of a unitary structure with the housing. The mouthpiece 6 is preferably configured so that a user may easily form an effective seal between the user's mouth and the mouthpiece. For example, in at least one embodiment, the mouthpiece 6 has a ridge for the user's teeth.

In at least one embodiment, the spirometer further comprises a filter in connection with the inlet 3. The filter may comprise, for example, an anti-bacterial filter. In a preferred embodiment, the mouthpiece 6 comprises the filter.

The second fluid opening 4 will be referred to as the outlet, although the direction of air flow through the opening will vary depending on the use of the spirometer (i.e. whether the user is inhaling or exhaling). In embodiments, the outlet 4 may comprise a single aperture or a series of apertures, such as a manifold. The outlet 4 may also include a protective mechanism, such as a shield or a screen, to prevent dust and debris from entering the inside of the housing.

The housing 2 may be produced by a number of methods, including for example, three-dimensional printing or injection molding. The housing 2 is preferably made out of a light-weight plastic material. As discussed in more detail below, the housing 2 may be produced so as to contain any of a number of engineered structures 18, 23, 24, each of which acts upon the fluid flow through the housing in a beneficial way in some embodiments.

The spirometer 1 also comprises a fluid flow sensor 5. The fluid flow sensor 5 comprises a piezoelectric material 7 oriented within the housing 2 to produce an electric signal in response to fluid flow through the housing.

Piezoelectric materials are materials that produce an electric signal in response to a mechanical stress. The electric signal produced by a piezoelectric material will be proportional to the magnitude of the mechanical stress. Any known piezoelectric material 7 is contemplated for use in the spirometer. In at least one embodiment, one or more polymers displaying piezoelectric properties are used as the piezoelectric material 7. For example, piezoelectric polyvinylidene fluoride, also known as PVDF, offers several distinct advantages over other piezoelectric materials. The term piezoelectric polyvinylidene fluoride as used herein refers to any polymer, copolymer, blend, or composite in which polyvinylidene fluoride is piezoelectrically active. Piezoelectric polyvinylidene fluoride materials include but are not limited to the beta phase of polyvinylidene fluoride (β-PVDF), the piezoelectrically active copolymer poly(vinylidenefluoride-co-trifluoroethylene) (PVDF-TrFE), and the piezoelectrically active copolymer poly(vinylidene-co-tetrafluoroethylene) (PVDF-TFE).

In some embodiments, the piezoelectric material 7 is a flexible piezoelectric film. For instance, the piezoelectric material 7 may be a flexible film of piezoelectric polyvinylidene fluoride.

In embodiments, the fluid flow sensor 5 is oriented so that fluid flow through the housing 2 produces oscillating stresses in the piezoelectric material 7. For example, embodiments of the spirometer 1 include a fluid flow sensor 5 that comprises a cantilever 8.

The cantilever 8 comprises an arm 10 having a first end 11 and a second end 12. The arm 10 is made up of a flexible member that comprises the piezoelectric material 7. In at least one embodiment, the piezoelectric material 7 is attached to a flexible base material along at least a portion of the arm 10. In at least another embodiment, the piezoelectric material 7, itself, functions as the flexible member of the arm 10. For example, the arm 10 may be made up of a flexible film of piezoelectric polyvinylidene fluoride. In at least one embodiment, the piezoelectric material 7 may be coated with a protective layer that protects the sensor from spittle and other elements. The protective layer may be in the form of a protective coating, film, laminate, or tape. The protective coating may be, for example, a potting urethane.

The cantilever also comprises an anchor 13. The anchor secures the cantilever arm 10 in a fixed location so that the arm flexes about an established flex point 14 in response to fluid flow through the housing 2. In embodiments, the anchor 13 secures at least the first end of the cantilever arm 11 in a fixed location. For example, the anchor 13 may securably connect the first end of the arm 11 to the housing 2. The connection may be either direct or indirect, so long as the first end of the cantilever is secured in a fixed location.

In some embodiments, the fluid flow sensor 5 may also comprise one or more stabilizers 15. The one or more stabilizers 15 are configured to reduce or prevent undesirable movement, such as sideways movement, of the cantilever arm 10 in response to fluid flow through the housing 2. By stabilizing the cantilever arm 10, unwanted contributions to the stresses on the piezoelectric material 7 during fluid flow through the housing 2 may be minimized. For example, the one or more stabilizers 15 may connect each side of the arm 10 to the housing 2. The connection between the arm 10 and the housing 2 is configured so that it reduces undesirable movement of the arm while at the same time not preventing flexing of the arm in the desired first and second directions in response to fluid flow through the housing.

In some embodiments, a stimulator 9 is located at the second end of the cantilever arm. When subjected to fluid flow, the stimulator 9 induces oscillating flexing of the cantilever arm 10. For example, the stimulator 9 is configured to produce vortex shedding on its downstream side. The vortex shedding causes the stimulator 9 to oscillate in a direction lateral to the fluid flow. The oscillation of the stimulator 9 causes the cantilever arm 10 to flex in an alternating manner between a first direction, as illustrated in FIG. 2, and a second direction, as illustrated in FIG. 3. This flexing induces oscillating stresses in the piezoelectric material 7, which produces an electric signal. Although the stimulator 9 illustrated in the Figures is a cylinder, the stimulator is not limited to any particular geometric shape or size.

The stimulator 9 may be made of any material. For example, the stimulator 9 may be made of a plastic material. In at least one embodiment, the stimulator 9 is made of the same material as the protective layer that protects the piezoelectric material 7. The stimulator 9 may be affixed to the second end of the cantilever arm or integrally formed with the cantilever arm 10. For example, the piezoelectric material 7 could be placed in a mold that defines the cantilever arm 10 and the stimulator 9. The mold may then be filled with a polymeric material that forms the protective layer and the stimulator 9, for example a urethane potting compound. Alternatively, the stimulator 9 may be 3D printed, injection molded, or created by dip coating.

The oscillating flexing of the cantilever arm 10 may also be induced in other ways. For example, in some embodiments, the spirometer 1 comprises one or more turbulence inducers 18. As fluid flows through the spirometer, a turbulence inducer 18 acts to shed vortices, creating turbulent flow that acts on the fluid flow sensor 5. A turbulence inducer 18 may be any engineered structure that acts to create turbulent flow. In some embodiments, the one or more turbulence inducers 18 may be integral with or directly molded into the housing 2.

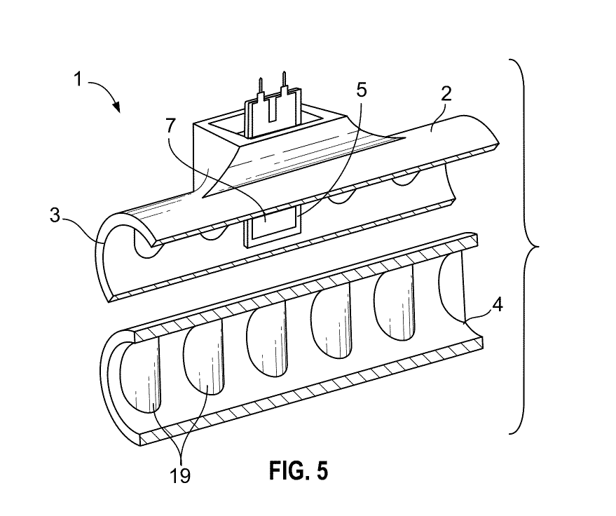

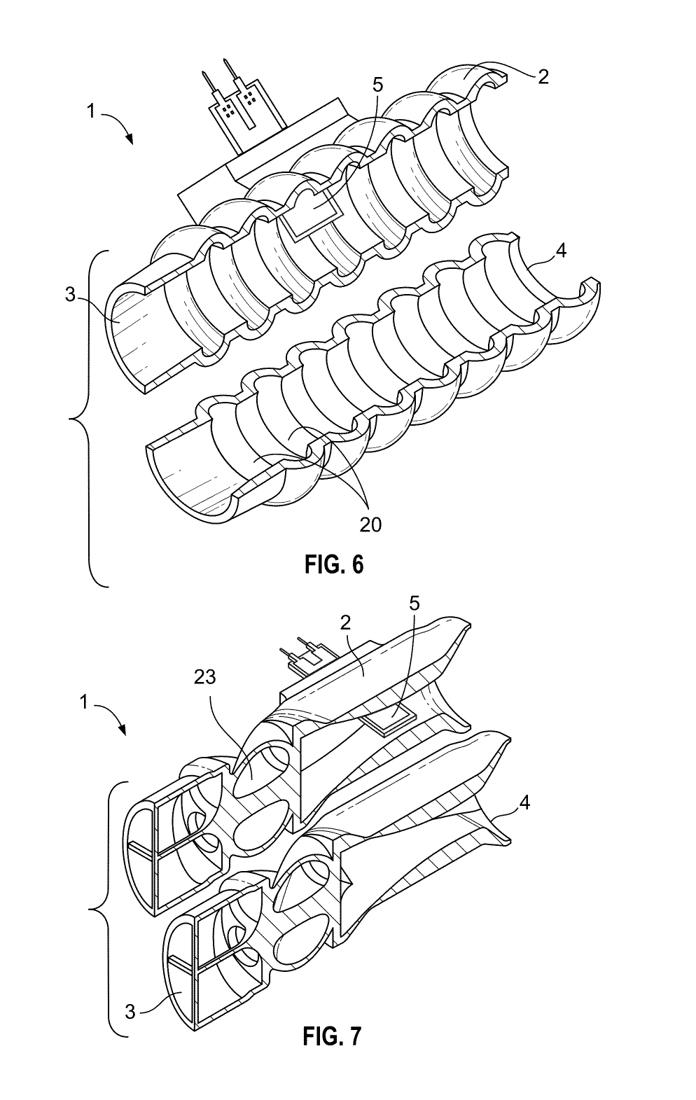

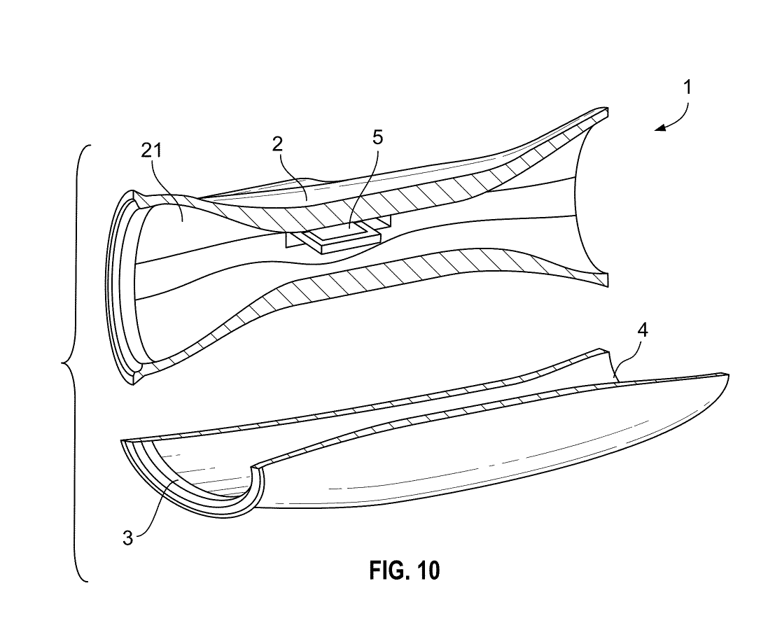

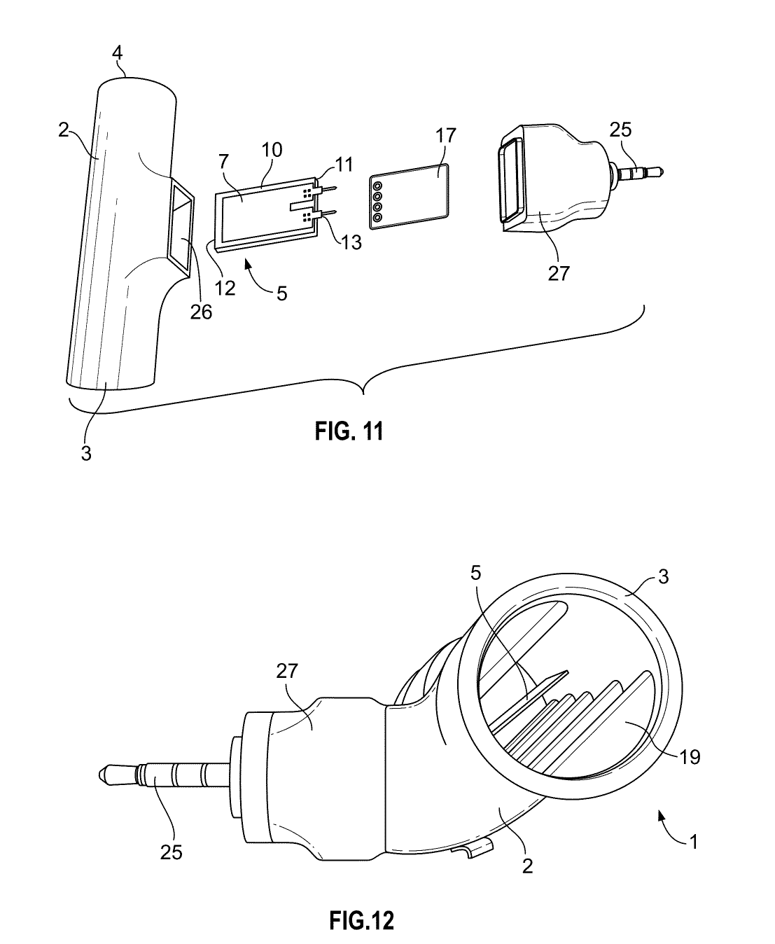

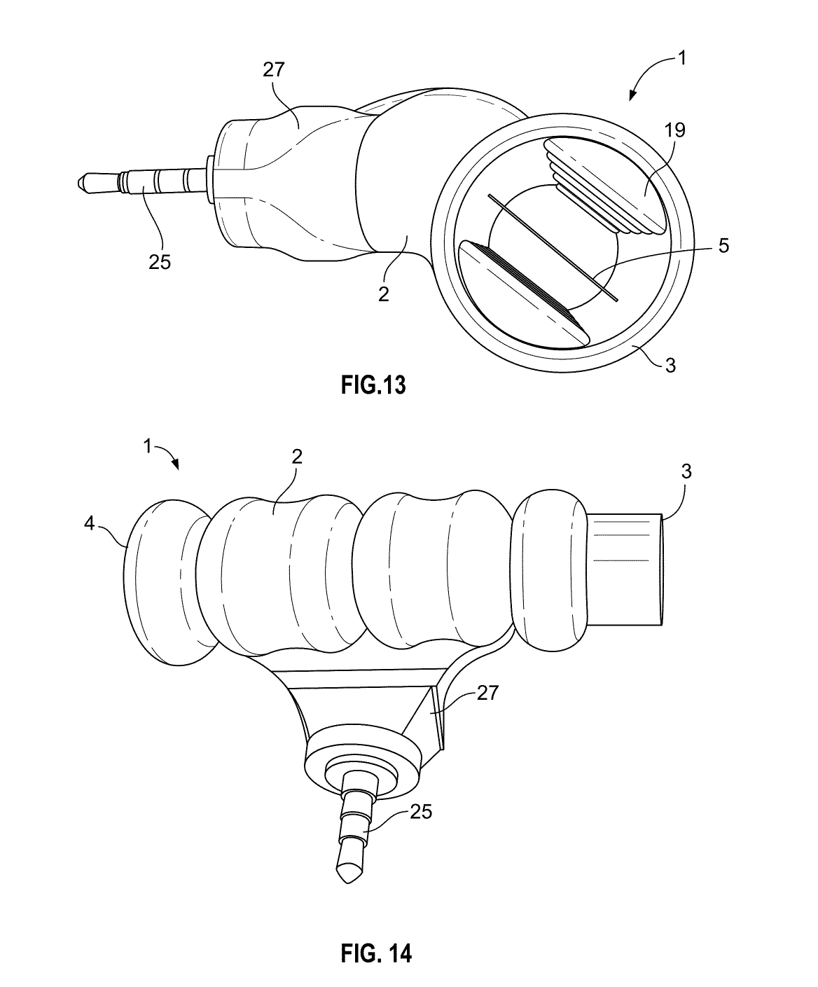

A turbulence inducer 18 may comprise any number of shapes. In some embodiments, the turbulence inducer 18 may comprise one or more columns 19. For example, in the embodiment illustrated in FIG. 5, the turbulence inducer 18 comprises a series of spaced-apart columns 19 at opposing sides of the housing. In some embodiments, the turbulence inducer 18 may comprise one or more cutaways, or inverted columns 20. In the embodiment illustrated in FIG. 6, for example, the turbulence inducer 18 comprises a series of spaced-apart cutaways 20 at opposing sides of the housing. The turbulence inducer 18 may also comprise a contoured wall 21, such as that illustrated in the embodiment of FIG. 10. The contour of the wall may take on many alternative arrangements. For example, in some embodiments, the housing may have a wall that is shaped to include a spiraled inner surface 22 in the region of the fluid flow sensor 5.

Because the turbulence inducer 18 creates fluid flow that is not parallel to the surface of the fluid flow sensor 5, for example the direction of flow between the first fluid opening 3 and the second fluid opening 4, the cantilever arm 10 may be caused to undergo oscillating flexing in response to fluid flow without the use of a stimulator 9. The cantilever arm 10 of the fluid flow sensor 5 must merely be located so as to be acted on by the vortices shed by the turbulence inducer 18.

In the spirometer 1 of the present invention, the electric signal produced by the piezoelectric material 7 corresponds with the rate of fluid flow through the housing 2. By corresponds, it is meant simply that the electric signal can be used to measure the rate of fluid flow through the housing. For example, the fluid flow sensor 5 is calibrated so that a particular fluid flow through the housing is known to correspond to an electric signal having particular characteristics, for example a particular magnitude. A particular electric signal may then be modified through a calibration equation to provide output that accurately represents the desired fluid flow data.

In some embodiments, the flow of fluid over the fluid flow sensor 5 produces a complex electric signal having a variety of frequencies. In this case, the spirometer 1 can be calibrated such that the magnitude of this signal corresponds with the fluid flow through the housing. There will, however, also be unwanted signals, or noise, that exists across the frequencies and the accuracy of the spirometer will be limited by the noise. Therefore, in other embodiments, the spirometer 1 is calibrated such that the amplitude of a particular frequency or the magnitude of a particular set or band of frequencies corresponds with the fluid flow through the housing. Using a frequency domain, such as a Fourier transform, the unwanted signals may be discarded and only the desirable frequencies measured. This enables the spirometer 1 to achieve a more accurate and precise measurement. For example, in some embodiments the amplitude of the signal at several predetermined frequencies may be combined and that magnitude may be compared against the total magnitude of the signal in order to produce information that precisely corresponds with and represents the fluid flow through the housing.

Accordingly, in some embodiments, the spirometer 1 is configured to produce a structured fluid flow, i.e. a flow having at least one predetermined frequency that interacts with the fluid flow sensor 5 to produce a signal that corresponds with the fluid flow through the housing. Structured flow may be produced by, for example, a turbulence inducer 18. By creating flow having at least one predetermined frequency, a turbulence inducer 18 creates fluid flow that acts in a specific measurable way on the fluid flow sensor 5. Thus, rather than measuring the response of the fluid sensor 5 to fluid flow over a large range of frequencies and converting the magnitude of that response to a fluid flow rate (in which the unwanted signals, i.e. noise, inherently reduces the accuracy and/or precision of the output), a turbulence inducer provides that the response of the fluid sensor to fluid flow only at particular, predetermined frequencies may be utilized to create the output data. By processing the signal generated by the fluid flow sensor, such as with a Fourier transform, the unwanted signals may be discarded before conversion to a fluid flow rate. Thus, the output data may be prepared using only the predetermined frequencies generated by the turbulence inducer 18. In this way, the spirometer 1 may be calibrated so that accuracy and precision of the output data is greatly increased.

In some embodiments, the spirometer may also be configured so that a characteristic flow of fluid over the sensor 5 is consistently achieved. In other words, the spirometer may be configured to prevent outside factors or variables from affecting the measurement provided by the sensor 5. For example, a user does not typically exhale into a spirometer 1 in a way that produces a consistent and repeatable flow. Rather, the exhaled air often deflects off any of a variety of surfaces, limiting the precision of many spirometers. For example, by simply tilting a spirometer, a user may exhale air into a spirometer in such a way that it deflects against a surface of the mouthpiece or spirometer. A user may also create turbulence simply by moving his or her tongue or lips during exhalation. All of this misdirected fluid flow can potentially cause a negative effect on a spirometer's accuracy of measurement. Thus, in some embodiments, it may be important that the spirometer 1 creates a consistently accurate measurement that is independent from outside factors.

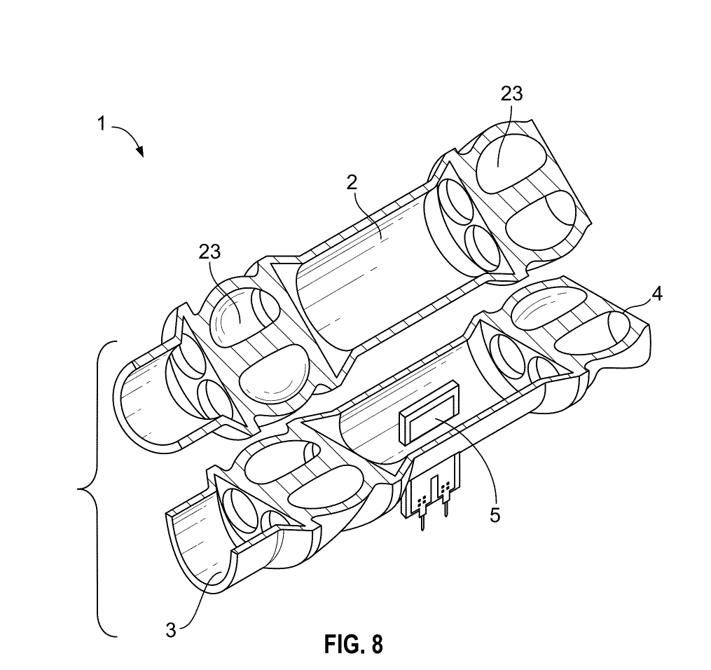

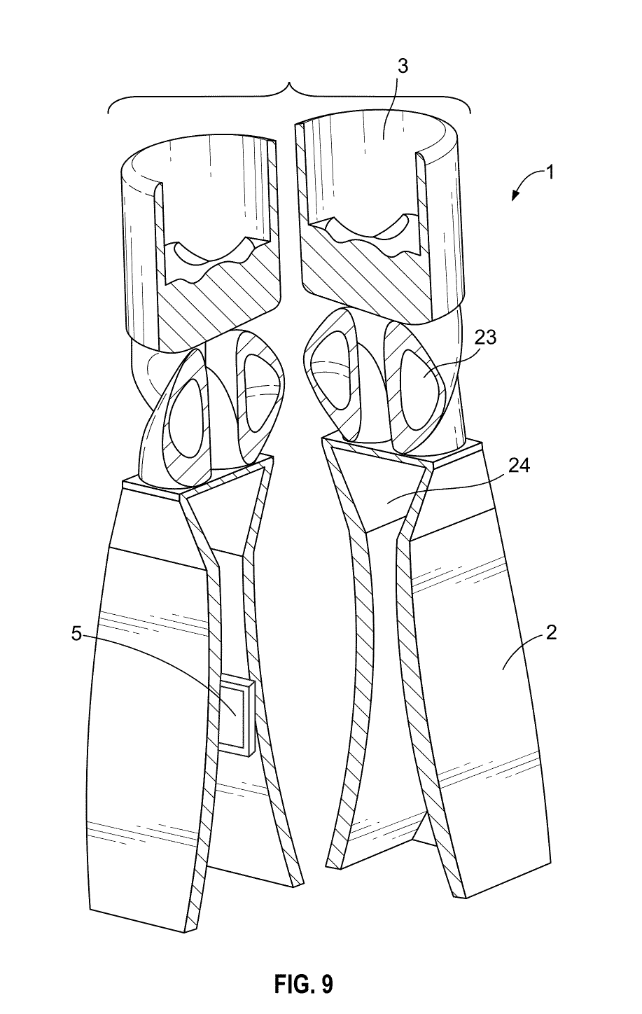

Accordingly, in some embodiments, the spirometer 1 is configured so as to condition the fluid flow prior to the fluid flow coming into contact with the sensor 5, thereby ensuring that a characteristic fluid flow over the sensor is consistently achieved. To achieve this, the spirometer may comprise a conditioner 23. A conditioner 23 acts on the fluid prior to its contact with the fluid flow sensor so that the fluid flow, including any misdirected fluid, is converted to a more consistent flow profile. A conditioner 23 may achieve this by acting upon the fluid flow in such a way as to prevent the fluid from having a straight path between the inlet 3 and the sensor 5. For example, the conditioner 23 may comprise two or more flow paths in a helical configuration, wherein the helical flow paths serve to rotate the air before it comes into contact with the fluid flow sensor 5. For example, the conditioner 23 illustrated in the embodiments shown in FIGS. 7, 8, and 9 comprises four tubes that rotates the air before it comes into contact with the fluid flow sensor 5. The embodiment shown in FIG. 9 comprises conditioners 23 on both sides of the fluid flow sensor 5 to ensure consistent measurements whether air is being inhaled or exhaled.

In other embodiments, the spirometer 1 may be configured to produce a substantially laminar flow of fluid over the sensor 5. By configuring the spirometer 1 to convert the air to a substantially laminar flow for at least the fluid flow path over the fluid flow sensor 5, embodiments of the spirometer 1 are capable of producing a consistently accurate response independent from outside factors.

In some embodiments, the spirometer 1 may also be configured to enhance the velocity of the fluid flow over the sensor 5. By increasing the velocity of the fluid flow over the sensor 5, the magnitude of the electrical signal is increased. This can be especially useful when measuring low flow rates of air being inhaled or exhaled. Accordingly, some embodiments of the spirometer 1 comprise a velocity enhancer 24. The velocity enhancer 24 may be molded into the housing or otherwise integral with the housing. For example, the velocity enhancer may comprise a narrowing section of the housing, as shown in the embodiment in FIG. 9.

There is, however, an upper bound to the amount of velocity enhancement that a spirometer may achieve before the oscillation of the sensor hits its resonance frequency, at which point the electric signal will no longer correspond to the fluid flow velocity. Thus, care must be taken to ensure that the velocity enhancement does not exceed the upper bound at which resonance of the fluid flow sensor occurs.

The spirometer 1 should also be carefully configured so as not to provide too much resistance to the flow of fluid between the inlet 3 and the outlet 4. If resistance within the spirometer 1 is overly high, the spirometer will no longer be capable of producing a representative measurement due to the user's lungs inherent action to combat the resistance. Accordingly, resistance within a particular spirometer design should be monitored. For example, velocity enhancers 24 are preferably located just before the fluid flow sensor 5, and the narrowed fluid pathway should not extend through too much of the housing 2. In the embodiment in FIG. 19, for example, the narrowed fluid pathway expands shortly after the fluid flow passes the fluid flow sensor 5. Care should also be taken to ensure that any conditioners 23 and/or turbulence inducers 24 do not overly increase the resistance of the spirometer 1.

Embodiments of the spirometer 1 are also configured to be coupled to an external display device 16. The display device 16 may be an external processing unit such as a personal computer or a smartphone. The term personal computer is meant to include but is not limited to desktop computers, laptop computers, tablets, and the like. The spirometer 1 may be configured to be coupled to an external display device 16 by a physical connection. For example, the spirometer 1 may be configured to be coupled to an external display device 16 through one or more coupling devices 25, such as a USB cable, a serial cable, a headphone cable, a specially configured cord, and combinations therein. Accordingly, embodiments of the spirometer 1 may comprise any of a USB cable, a serial cable, a headphone cable, and combinations thereof. The spirometer 1 may also be configured to be coupled to an external display device 16 by a wireless connection. For example, the spirometer 1 may be configured to be coupled to an external display device 16 using Bluetooth technology, wifi technology, infrared transmission, or fiber optics. Accordingly, embodiments of the spirometer 1 may comprise a Bluetooth transmitter. In at least one embodiment, the display device 16 may be integral with the spirometer 1. For example, the spirometer 1 may comprise an LCD display, an LED display, an organic LED display, a raised touch pad, or combinations therein.

Embodiments of the spirometer 1 also comprise a signal modification unit 17, which is operable to modify the electric signal. The signal modification unit 17 may be operable to amplify the signal, to condition the signal, to convert the signal from analog to digital, or a combination of the above. Conditioning of the signal may comprise, for example, full wave rectification, frequency conversion, and the like. In at least one embodiment, the signal modification unit 17 comprises a conditioning circuit. The exact functions of the signal modification unit 17 may depend on the manner or manners by which the spirometer 1 is configured to be coupled to a display device 16.

For example, in at least one embodiment where the spirometer 1 is configured to be coupled to an external display device 16 through a headphone cable, the signal modification unit 17 comprises a conditioning circuit that operates to amplify the signal, pass the signal through a full wave rectifier, and convert the frequency of the signal. As another example, in at least one embodiment where the spirometer 1 is configured to be coupled to an external display device 16 through a Bluetooth connection, the signal modification unit comprises an analog to digital convertor. In another embodiment, the signal modification unit 17 may simply operate to amplify the signal and pass it into a display device such as a smartphone as an audio waveform which can be picked up by the display device's microphone pickup.

Embodiments of the present invention are also directed to a method for measuring lung performance by inhaling or exhaling into the spirometer 1 of at least one embodiment of the invention. In this method, a spirometer 1 comprising a piezoelectric material 7 is provided and air is inhaled or exhaled into the device such that the flow of air in the spirometer acts upon the piezoelectric material to create an electric signal. The magnitude of the electric signal corresponds to the velocity or volume of air inhaled or exhaled into the spirometer. The magnitude of the electric signal may be measured and that information converted into output data that provides a user with information relating to the user's lung performance. For instance, the sum of the amplitudes at several predetermined frequencies may be measured in proportion to the total magnitude of the electric signal and that information may be converted into output data that reflects one or more of a user's lung performance parameters.

In some embodiments, the conversion of the electric signal into output data may also take into account other factors, such as the temperature, the humidity, or a combination of the two. For example, the spirometer or the external display device may comprise a temperature sensor, a humidity sensor, or both. The measurement from one or both of these sensors may thus be utilized to provide output data having increased accuracy and precision.

In various embodiments, the output data may be displayed on a personal computer or smartphone and the lung performance data may be tracked over a period of time. For instance, at least one of the electric signal and the output data may be transmitted to an external display, such as a personal computer or a smartphone using either a physical connection or a wireless connection.

The output data may include raw data, such as liters or liters per second. Output data may also include the test result as a percent of the predicted values for a patient of similar characteristics (height, age, sex, weight, etc.). Output data may also include graphical data. For example, output data may comprise a volume-time curve, showing volume along the Y-axis and time along the X-axis; a flow-volume loop, which graphically depicts the rate of airflow on the Y-axis and the total volume inhaled or exhaled on the X-axis; or any combination thereof.

A spirometer 1 in accordance with embodiments of the present invention may be used to measure and display as output data any of a number of lung performance parameters, including but not limited to, vital capacity (VC), forced vital capacity (FVC), forced expiratory volume (FEV) at timed intervals such as the FEV1 (one second) test, forced expiratory flow (FEF) such as FEF 25-75, peak expiratory flow (PEF), maximum breathing capacity, and combinations thereof.

In some embodiments, the personal computer or smartphone may comprise an application that is used to perform any or all of the following: track or monitor the output data over a period of time or a number of uses, analyze the output data to provide additional lung performance information, display the output data graphically, interface with other devices for offsite review or interpretation, and combinations thereof.

Embodiments of the present invention provide a spirometer 1 that assesses lung function by measuring the characteristics, such as the magnitude, of electric signals produced by the flow of fluid, such as inhaled or exhaled air, against a piezoelectric material 7. The piezoelectric material 7 used in embodiments of the spirometer 1 is able to detect small variations in air flow in order to provide a precise measurement. Accordingly, the spirometer 1 of embodiments of the present invention provides a more sensitive and precise measurement than conventional spirometers, especially those currently configured for home use. Additionally, because the fluid flow sensor 5 offers little to no resistance, the spirometer 1 of embodiments of the present invention has a low turn-on velocity, i.e. it requires little air flow to reach a minimum value at which detection and measurement may occur. Both of these effects offer significant advantages over conventional spirometer technology.

Embodiments of the present invention also provide a spirometer 1 having an improved construction that renders the spirometer durable and economical compared to conventional devices. For example, the sensor 5, which comprises the piezoelectric material 7, produces the electric signal that is converted into output data. Thus, unlike conventional spirometers, the spirometer 1 of embodiments of the present invention does not require a conversion of the measurement parameter to an electric signal. This provides economic advantages in comparison to conventional spirometers by reducing the number of components that are required in the device. Embodiments of the present invention also provide a spirometer 1 that contains few moving parts. This renders the spirometer 1 more durable and economical than many conventional spirometers, making it particularly suitable for home use. The lack of moving parts also makes use of the spirometer 1 straightforward and easy. For example, the spirometer 1 need not be positioned at any particular angle to obtain an accurate measurement, as is the case with some turbine-based spirometers.

Embodiments of the present invention also provide a spirometer 1 that has increased portability over conventional spirometers. The piezoelectric-based sensor 5 may be very small and requires little in the way of additional components. Accordingly, the spirometer 1 may be configured to fit in a purse, briefcase, or messenger bag. Alternatively, the spirometer may be configured to fit in a clothing pocket, such as a standard pants pocket. Alternatively, the spirometer 1 may be configured to fit in a case for a smartphone or portable media device. Alternatively, the spirometer 1 may be configured to be affixed to a user, such that it can be used in a hands-free manner. For example, the spirometer 1 may be incorporated into face masks, scuba breathing tubes, or clothing such as high-performance running clothing. In this way, the spirometer 1 could be used to monitor lung performance by an athlete during athletic activity, e.g. by a long distance runner during running.

Embodiments of the spirometer 1 may be configured to have a length of less than 7 inches, alternatively embodiments of the spirometer may be configured to have a length of less than 6 inches, alternatively embodiments of the spirometer may be configured to have a length of less than 5 inches, alternatively embodiments of the spirometer may be configured to have a length of less than 4 inches, alternatively embodiments of the spirometer may be configured to have a length of less than 3 inches. Embodiments of the spirometer 1 may also be configured to have an outermost housing diameter of less than 2 inches, alternatively embodiments of the spirometer may be configured to have a outermost housing diameter of less than 1.5 inches, alternatively embodiments of the spirometer may be configured to have an outermost housing diameter of less than 1.25 inches, alternatively embodiments of the spirometer may be configured to have an outermost housing diameter of less than 1 inch.

Because embodiments of the spirometer 1 of the present invention are particularly effective, economical, durable, portable, and easy to use, it is contemplated that embodiments of the spirometer may bring about new spirometer use in the home for the tracking of lung function and/or the improvement of lung performance. For example, it is contemplated that embodiments of the spirometer 1 may find particular use by athletes, runners, bikers, musicians, singers, smokers, ex-smokers, children, and the like.

For example, embodiments of the spirometer 1 could be used, such as by any of the above, to improve lung function, e.g. as an incentive spirometer. It is especially contemplated that embodiments of the spirometer 1 could be used in connection with an “app” or a computer program to track improvements in lung performance over time. The “app” or program could provide incentives well beyond those of conventional incentive spirometers. For example, the “app” or computer program could use animations, games, and the like to incentivize use of the spirometer to improve lung performance.

Embodiments of the spirometer 1 could also be used by an individual at home to monitor various lung performance attributes. For example, the spirometer 1 could be used to produce output data comprising any of a number of lung performance parameters. The output data could then be made available to a health care professional, if desired. This could save unnecessary visits to the office of a health care professional or hospital. Embodiments of the spirometer 1 could also be used in coordination with an “app” or computer program that guides the user through the various testing steps, for example by telling the user when to inhale and when to exhale. For instance, the spirometer 1 could be linked with the app to ensure that an accurate measurement is taken.

Embodiments of the spirometer 1 are also contemplated for use in health care settings, as they also provide an improvement over conventional spirometers that are used by health care professionals.

The design and functions of a particular spirometer 1 in accordance with embodiments of the present invention may be adjusted according to its intended use. For example, a spirometer 1 that is intended for use in a health care setting may be configured to have a different design or may be programmed to provide different output data than a spirometer 1 that is intended for home use. Similarly, a spirometer 1 that is configured for improving lung function may be programmed to provide different output data than one that is configured for lung performance monitoring. In this manner, a spirometer 1 in accordance with various embodiments of the present invention may be designed for general use or for use by a specific audience.

### Example 1

To test that a spirometer according to embodiments of the present invention would work for its intended purpose, an initial prototype was built. A sheet of piezoelectric PVDF-TrFE was provided by Measurement Specialties and encapsulated in a urethane compound to create a cantilever arm. A stimulator, which consisted of a hollow plastic cylinder, was attached to one end of the cantilever arm. The other end of the cantilever arm was then anchored by compression fitting to the housing. The housing consisted of a PVC (polyvinyl chloride) tube that was cut to a desired length. Electrical leads were connected to the piezoelectric material. Specifically, a first wire was soldered to a first side of the PVDF-TrFE sheet and a second wire was soldered to the second side of the PVDF-TrFE sheet. By doing so, the electrical leads were able pick up the electric signal produced by the piezoelectric material as it flexed in either direction. The electrical leads were routed through the housing and connected to an oscilloscope. The spirometer was tested by providing an air flow into one end of the housing, wherein the air flow was provided at varying degrees of force, e.g. low, medium, and high. In each instance, the oscilloscope displayed the oscillating electric signal from the fluid flow sensor. The magnitude of the oscillating electric signal was shown to correspond to the degree of force of the air flow at each setting.

### Example 2

After the initial prototype testing, a variety of spirometer devices were built. Using a 3-D printer, housings having a variety of designs were prepared. For example, a typical spirometer embodiment was designed to have a length of about 3 inches and a housing diameter of about 1 inch. Next, a fluid flow sensor 5 was inserted into a housing 2 at a desired location, such as through a port 26 that was designed in the bottom of the housing.

The fluid flow sensor 5 was prepared by encapsulating a sheet of piezoelectric PVDF-TrFE from Measurement Specialties in a urethane compound to create a cantilever arm 10. Electrical leads were connected to the piezoelectric material. Specifically, a first wire was soldered to a first side of the PVDF-TrFE sheet and a second wire was soldered to the second side of the PVDF-TrFE sheet. By doing so, the electrical leads were able pick up the electric signal produced by the piezoelectric material as it flexed in either direction.

Before being inserted into the housing, the fluid flow sensor was connected with the signal modification circuitry 17 and the coupling device 25. For the prototypes made in accordance with this Example, a microphone plug was used as the coupling device 25 and the sensor 5, circuitry 17, and microphone plug were soldered together. The circuitry was also coated with a polymer to protect it from potential fouling, such as due to moisture. The circuitry and the microphone plug were housed in a circuitry enclosure 27 that was designed to fit snugly with the port on the housing 26. This enabled the spirometer 1 to be sealed by connecting the circuitry enclosure 27 with the port on the housing 26 using an adhesive. The components of a prototype made in accordance with this Example can be seen in FIG. 11.

### Example 3

The spirometers made in accordance with Example 2 were next calibrated and tested to determine if they could consistently produce accurate fluid flow measurements. Using a controllable source of fluid flow, in this case a vacuum to pull air, a calibration system was prepared. A commercially available hot wire anenmometer, Omega Engineering® model HHF-SD1, was mounted in line with the vacuum and valve to control the air flow speed. A spirometer built in accordance with embodiments of the present invention was also mounted in line with the vacuum and valve to control the air flow speed. Controlling the air flow at various velocities between 0 and 10 liters per second, data points were recorded for (a) the fluid flow velocity as measured by the commercial sensor and (b) the signal output of the spirometer in accordance with embodiments of the present invention. An equation was derived to fit the curve generated by the data points. This curve was then used to calibrate the signal output of the invention to the known flow rate as measured by the hot wire anenmometer. Then, the calibration of the spirometer in accordance with embodiments of the present invention was tested using a calibrated 3L syringe and it was determined that the volume measured by the spirometer was accurate.

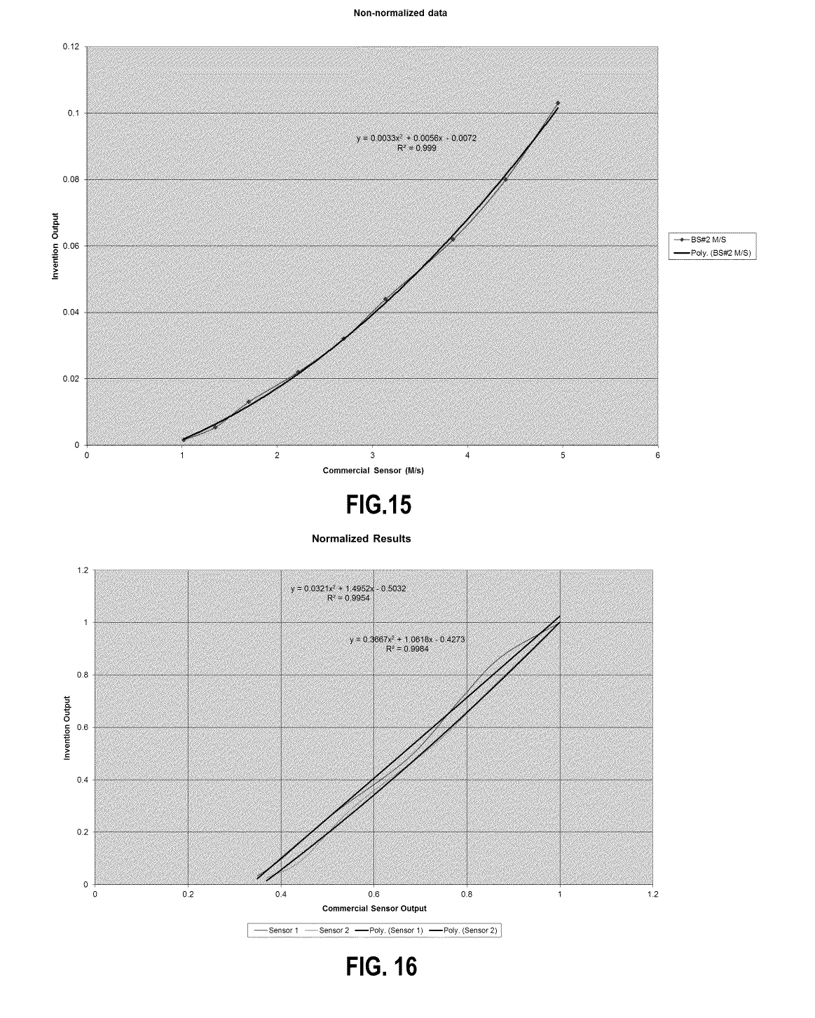

As shown in FIG. 15, spirometers according to embodiments of the present invention can be calibrated to provide a fluid flow measurement having a degree of confidence of at least 99.9% when compared against the highly accurate Omega Engineering® model HHF-SD1. The calibration data may also be normalized to produce a calibration equation such as those shown in FIG. 16. The calibrations of the two spirometer embodiments shown in FIG. 16 were achieved to a degree of confidence of 99.5 for the device labeled “Sensor 1” and 99.8% for the device labeled “Sensor 2”.

Embodiments of the spirometer may be calibrated, such as described above, to provide a fluid flow measurement having an accuracy of greater than 99.5%; alternatively the spirometer may be calibrated, such as described above, to provide a fluid flow measurement having an accuracy of greater than 99.6%; alternatively the spirometer may be calibrated, such as described above, to provide a fluid flow measurement having an accuracy of greater than 99.7%; alternatively the spirometer may be calibrated, such as described above, to provide a fluid flow measurement having an accuracy of greater than 99.8%; alternatively the spirometer may be calibrated, such as described above, to provide a fluid flow measurement having an accuracy of greater than 99.9%.

Because embodiments of the spirometer may be configured to have a very low turn-on velocity, the spirometer may be capable of measuring very low fluid flows. In some embodiments, the spirometer can be configured and calibrated to measure fluid flows at least as low as 0.05 liters per second, alternatively at least as low as 0.01 liters per second, alternatively at least as low as 0.005 liters per second, alternatively at least as low as 0.001 liters per second. In some embodiments, the spirometer may also be configured and calibrated to measure fluid flows at least as high as 14 liters per second, alternatively at least as high as 17 liters per second, alternatively at least as high as 20 liters per second, alternatively at least as high as 25 liters per second.

The sampling frequency of embodiments of the spirometer may be much higher than that of conventional spirometers. For example, in some embodiments, the spirometer may have a sampling frequency of greater than 40 kHZ, alternatively greater than 60 kHZ, alternatively greater than 80 kHZ, alternatively greater than 90 kHZ, alternatively greater than 100 kHZ, alternatively greater than 110 kHZ, alternatively greater than 120 kHZ, alternatively greater than 130 kHZ, alternatively greater than 140 kHZ, alternatively greater than 150 kHZ.

It can be seen that the described embodiments provide a unique and novel spirometer that has a number of advantages over those in the art. While there is shown and described herein certain specific structures embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A spirometer comprising
a. a housing having a first fluid opening and a second fluid opening, and
b. a fluid flow sensor comprising a piezoelectric material oriented within the housing to produce an electric signal in response to fluid flow through the housing,
wherein the spirometer is configured so that fluid flow through the housing produces oscillating stresses in the piezoelectric material, and
wherein the electric signal has a magnitude that corresponds with the rate of fluid flow through the housing.

2. The spirometer of claim 1, wherein the fluid flow sensor comprises
a. a cantilever comprising the piezoelectric material, and
b. a stimulator,
wherein the stimulator is configured to induce flexing of the cantilever in response to fluid flow through the housing, and
wherein the flexing brings about said oscillating stresses in the piezoelectric material.

3. The spirometer of claim 1, wherein the fluid flow sensor comprises
a. a cantilever comprising the piezoelectric material, and
a. a turbulence inducer,
wherein the turbulence inducer is configured to induce flexing of the cantilever in response to fluid flow through the housing, and
wherein the flexing brings about said oscillating stresses in the piezoelectric material.

4. The spirometer of claim 1, wherein the spirometer is configured to produce a structured flow.

5. The spirometer of claim 1, wherein the cantilever consists of a flexible piezoelectric film and a protective coating.

6. (canceled)

7. The spirometer of claim 1, wherein the piezoelectric material comprises piezoelectric polyvinylidene fluoride.

8. (canceled)

9. The spirometer of claim 1, further comprising a signal modification unit for modifying the electric signal.

10. The spirometer of claim 1, wherein the fluid flow sensor is configured to be coupled to a display device.

11. The spirometer of claim 10, wherein the spirometer is configured to be coupled to a display device by a physical connection.

12. The spirometer of claim 10, wherein the spirometer is configured to be coupled to a display device by a wireless connection.

13. The spirometer of claim 10, wherein the display device is a smartphone.

14. The spirometer of claim 10, wherein the display device is a personal computer.

15.-32. (canceled)

33. The spirometer of claim 1, wherein the spirometer is configured to condition the fluid flow prior to the fluid flow coming into contact with the sensor.

34. The spirometer of claim 1, wherein the spirometer is configured to enhance the velocity of the fluid flow over the sensor.

35. The spirometer of claim 1, wherein the spirometer is calibrated to provide the rate of fluid flow through the housing with an accuracy of greater than 99.8 percent.

36. The spirometer of claim 1, wherein the spirometer is configured to measure fluid flows as low as 0.01 liters per second.

37. The spirometer of claim 1, wherein the spirometer is configured to have a sampling frequency of greater than 90 kHZ.

38.-43. (canceled)

44. The method of claim 1, wherein the magnitude that corresponds with the rate of fluid flow through the housing is the sum of the amplitudes at multiple predetermined frequencies.

45. The method of claim 44, wherein the sum of the amplitudes at multiple predetermined frequencies is compared against the total magnitude of the electric signal.

46. A spirometer comprising
a. a housing having a first fluid opening and a second fluid opening;
b. a fluid flow sensor oriented within the housing, the fluid flow sensor comprising a cantilever and a piezoelectric material; and
c. a turbulence inducer;
wherein the turbulence inducer is configured to induce flexing of the cantilever in response to fluid flow through the housing, the flexing bringing about oscillating stresses in the piezoelectric material to produce an electric signal; and
wherein the spirometer is configured to produce a structured flow such that the magnitude of the electric signal at a particular set of frequencies closely corresponds with the rate of fluid flow through the housing.
